(19) United States
(12) Patent Application Publication
(10) Pub. No.: US 2009/0069878 A1
(43) Pub. Date: Mar. 12, 2009

(54) BIFURCATION POST-DILATATION BALLOON AND METHODS

(75) Inventors: Jan Weber, Maastricht (NL); James M. Anderson, Fridley, MN (US); Karl Jagger, Deephaven, MN (US); James Lee Shippy, Wilmington, NC (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc., Maple Grove, MN (US)

(21) Appl. No.: 12/184,607

(22) Filed: Aug. 1, 2008

Related U.S. Application Data
(60) Provisional application No. 60/968,228, filed on Aug. 27, 2007.

(51) Int. Cl. A61F 2/84; A61M 25/10

(52) U.S. Cl. 623/1.11; 604/99.01

(57) ABSTRACT

A catheter assembly includes a balloon member and a balloon restricting arrangement. The balloon member includes a proximal portion, a distal portion and a circumferential bulge portion. The circumferential bulge portion extends around a circumference of the balloon member and is positioned at a location between the proximal and distal portions of the balloon member. The balloon restricting arrangement includes a proximal portion extending around the proximal portion of the balloon member and a distal portion extending around the distal portion of the balloon member to restrict an inflated size of the proximal and distal portions of the balloon member. The proximal and distal portions of the balloon restricting arrangement are spaced apart axially, and the circumferential bulge portion extends radially outward therebetween. The circumferential bulge portion inflates to a maximum inflated dimension that is greater than an inflated dimension of the proximal and distal portions of the balloon restricting arrangement.

### CROSS REFERENCE TO RELATED APPLICATIONS

This application claims priority to U.S. Provisional Application Ser. No. 60/968,228 filed on Aug. 27, 2007, entitled APPARATUS FOR CONTROLLING AND METERING FLUID FLOW, the disclosure of which is incorporated by reference herein in its entirety.

### TECHNICAL FIELD

This disclosure generally relates to catheter assemblies for use in treating vessel bifurcations, and more particularly relates to post-dilatation balloons for treatment of vessel bifurcations.

### BACKGROUND

Catheters are used with stents and inflatable structures to treat conditions such as strictures, stenoses, and narrowing in various parts of the body. Various catheter designs have been developed for the dilatation of stenoses and to deliver and deploy stents at treatment sites within the body.

Stents are typically intraluminally placed by a catheter within a vein, artery, or other tubular shaped body organ for treating conditions such as, for example, occlusions, stenoses, aneurysms, dissections, or weakened, diseased, or abnormally dilated vessels or vessel walls, by expanding the vessels or by reinforcing the vessel walls. Once delivered, the stents can be expanded using one or more inflatable members such as balloons. Stents can improve angioplasty results by preventing elastic recoil and remodeling of the vessel wall and treating dissections in blood vessel walls caused by balloon angioplasty of coronary arteries. Stents can also be used as a drug delivery medium for treatment of damaged portions of a vessel.

While conventional stent technology is relatively well developed, stent technologies related to treatment of the region of a vessel bifurcation are still being developed.

### SUMMARY

The present disclosure generally relates to catheter assemblies for treatment of vessel bifurcations. Although alternatives are possible, an example catheter assembly generally includes a balloon member and a balloon restricting arrangement. The balloon member includes a proximal portion, a distal portion and a circumferential bulge portion. The circumferential bulge portion extends around a circumference of the balloon member and is positioned at a location between the proximal and distal portions of the balloon member. The balloon restricting arrangement includes at least a proximal portion extending around at least a portion of the proximal portion of the balloon member, and a distal portion extending around at least a portion of the distal portion of the balloon member. The balloon restricting arrangement restricts an inflated size of the proximal and distal portions of the balloon member. The proximal and distal portion of the balloon restricting member are spaced apart axially, wherein the circumferential bulge portion extends radially outward in the space defined between the proximal and distal portions of the balloon restricting member. The circumferential bulge portion inflates to a maximum inflated dimension that is greater than a maximum dimension of the proximal and distal portions of the balloon restricting member.

There is no requirement that an arrangement or method include all features characterized herein to obtain some advantage according to this disclosure.

### DESCRIPTION OF THE DRAWINGS

FIG. 1 is a schematic side view of an example catheter assembly in accordance with principals of the present disclosure and the balloon member in the uninflated state.

FIG. 2 is a schematic side view of the catheter assembly shown in FIG. 1 with the balloon member in an inflated state.

FIG. 3 is a schematic front view of the catheter assembly shown in FIG. 2.

FIG. 4 is a schematic side view of another example catheter assembly in accordance with principals of the present disclosure with the balloon member in the uninflated state.

FIG. 5 is a schematic side view of the catheter assembly shown in FIG. 4 with the balloon member in an inflated state.

FIG. 6 is a schematic end view of the catheter assembly shown in FIG. 5.

FIG. 7 is a schematic side view of another example catheter assembly in accordance with principals of the present disclosure with the balloon member in the uninflated state.

FIG. 8 is a schematic side view of the catheter assembly shown in FIG. 7 with the proximal and distal portions of the balloon member in an inflated state.

FIG. 9 is a schematic side view of the catheter assembly shown in FIG. 7 with a circumferential bulge portion of the balloon member also in an inflated state.

FIG. 10 is a schematic end view of the catheter assembly shown in FIG. 9.

FIG. 11 is a schematic side view of another example catheter assembly in accordance with principals of the present disclosure and the balloon member in an uninflated state.

FIG. 12 is a schematic side view of the catheter assembly shown in FIG. 11 with the balloon member in an inflated state.

FIG. 13 is a schematic end view of the catheter assembly shown in FIG. 12.

FIG. 14 is a schematic side view of an example restricting member in accordance with principals of the present disclosure having a plurality of flexing grooves formed therein.

FIG. 15 is a schematic side view of another example restricting member in accordance with principals of the present disclosure and including a marker member positioned thereon.

FIG. 16 is a schematic end view of the example restricting member shown in FIG. 15.

FIG. 17 is a schematic side view of an example stent delivery catheter system positioned at a vessel bifurcation and the balloon member in an uninflated state.

FIG. 18 is a schematic side view of the stent delivery system shown in FIG. 7 with the balloon member in an inflated state to expand the stent into engagement with the main vessel of the vessel bifurcation.

FIG. 19 is a schematic side view of the catheter assembly shown in FIG. 4 positioned at a vessel bifurcation and in alignment with the branch vessel of the vessel bifurcation.

FIG. 20 is a schematic side view of the catheter assembly shown in FIG. 19 with the balloon member in an inflated state.

FIG. 21 is a schematic side view of a post dilation balloon positioned extending through a side opening in the stent and into the branch vessel.

FIG. 22 is a side view of an example stent in accordance with the present disclosure, the stent include high and low density portions.

FIG. 23 is a schematic side view of balloon catheter for a stent delivery system constructed according to principles of the present disclosure, wherein the balloon includes a circumferential bulge portion.

FIG. 24 is a schematic side view of the balloon catheter shown in FIG. 1 with the balloon in an inflated stated.

FIG. 25 is a cross-sectional view of a balloon catheter shown in FIG. 2 taken along cross-sectional indicators 3-3.

FIGS. 26-32 illustrate steps of an example method of treating a vessel bifurcation using the balloon catheter assembly of FIGS. 23-25 and a post-dilation balloon catheter.

FIGS. 33-37 illustrate steps of another example method of treating a vessel bifurcation using the balloon catheter assembly shown in FIGS. 23-25.

FIG. 38 is a schematic perspective view of an example balloon mold for use in forming the balloon catheter assembly shown in FIGS. 23-25.

FIG. 39 is a top view of the balloon mold shown in FIG. 38.

FIG. 40 is a cross-sectional view of the balloon mold shown in FIG. 38 taken along cross-sectional indicators 40-40.

FIG. 41 is a close-up view of the balloon bulge portion of the balloon mold shown in FIG. 38.

### DETAILED DESCRIPTION

This disclosure relates to bifurcation treatment systems, catheter assemblies, and related methods of treating bifurcations in a patient's body. The term bifurcation means a division location from one unit into two or more units. Generally, two types of bifurcations of a body organ include: 1) a main tubular member defining a main lumen and a branch tubular member defining a branch lumen that extends or branches off from the main tubular member, wherein the main and branch lumens are in fluid communication with each other, and 2) a primary or main member defining a primary or main lumen (also referred to as a parent lumen) that splits into first and second branch members defining first and second branch lumens. The term lumen means the cavity or bore of a tubular structure such as a tubular organ (e.g., a blood vessel).

An example bifurcation is a vessel bifurcation that includes a continuous main vessel and a branch vessel, wherein the vessels define a main lumen and a branch lumen, respectively that are in fluid communication with each other. Alternatively, a vessel bifurcation can include a parent vessel that divides into first and second branch vessels, wherein the vessels define a parent lumen and first and second branch lumens, respectively, which lumens are all in fluid communication with each other.

Example applications of the inventive principles disclosed herein include cardiac, coronary, renal, peripheral vascular, gastrointestinal, pulmonary, urinary, and neurovascular systems. The catheter assemblies, systems and methods disclosed herein can be used for locating a branch vessel of the vessel bifurcation and for placement of a stent relative to the vessel bifurcation for treatment of the vessel bifurcation.

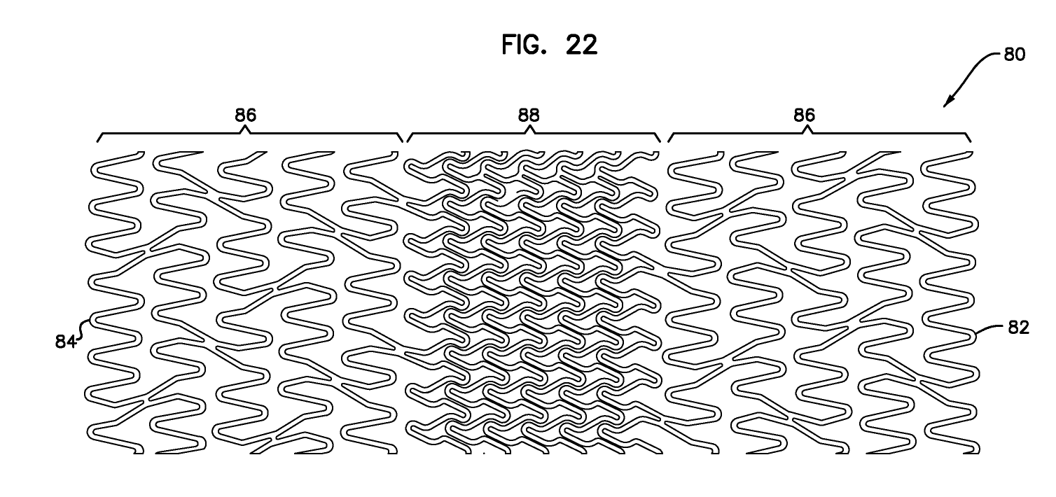

One way of treating a vessel bifurcation is to place a conventional stent in the main vessel of the vessel bifurcation and extend some of the struts of the stent as far as possible into the branch vessel of the vessel bifurcation to support the ostium of the vessel bifurcation. Some stent designs such as the stent construction shown in FIG. 22 include a plurality of longer free strut structures in the middle section of the stent. These longer free strut structures are able to extend further into the branch vessel as compared to struts at distal and proximal portions of the stent.

Even with such new stent designs, the stent structure that protrudes into the branch vessel from within the main vessel typically requires a certain balloon construction to move the stent structure into the branch vessel. One such balloon construction is a “submarine” type balloon that includes a bulge portion positioned at a particular circumferential location at a point along a length of the main balloon. The bulge portion must be aligned both radially and axially with the opening into the branch vessel. Inflation of the bulge portion when so aligned can help extend the stent struts into the branch vessel. The precise axial and radial alignment typically necessary for use of such submarine balloons can cause some difficulty in properly treating the vessel bifurcation.

Another option for extending the stent structure into the branch vessel is to use a balloon having a maximum inflated dimension that is greater than the minimum internal dimension of the main vessel. Inflation of such a balloon will cause the stent to bulge or otherwise expand into the branch vessel. However, the enlarged size of the balloon and resulting expanded size of the stent at locations proximal and distal of the opening into the branch vessel can cause undesired stress in those portions of the main vessel proximal and distal of the opening into the branch vessel. Such undesirable vessel expansion effects can arise whether using a compliant, semi-compliant or non-compliant balloon structure.

The following description with reference to the attached figures describe various balloon arrangements that provide bulging of the stent structure into the branch vessel without imposing undo stress in the main vessel wall at locations distal and proximal of the opening into the branch vessel. Some arrangements additionally provide for reduced stress in the main vessel wall opposite the opening into the branch vessel. Furthermore, the balloon arrangements described with greater detail below with reference to the figures can provide extension of the stent structure into the branch vessel without the need for radial positioning of any feature of the balloon relative to the opening into the branch vessel as long as a specified portion of the balloon is axially aligned relative to the opening into the branch vessel.

One feature common to some of the catheter assemblies described herein is a circumferentially arranged bulge portion of the balloon that extends further radially than proximal and distal portions of the balloon. This bulge portion typically extends around an entire circumference of the balloon member. In some arrangements, the bulge portion can extend less than completely around the entire circumference of the balloon member while still extending around substantially all of the circumference of the balloon member.

The bulge portion of the balloon can be formed in various ways using a variety of structures. Some example bulge portions are now described.

The example catheter assembly shown and described with reference to the attached figures are typically referred to as post dilation balloon catheter assemblies. A post dilation catheter assembly is typically used in a vessel after a stent has already been expanded into engagement with the vessel wall. In the case of treating a vessel bifurcation, a post dilation balloon catheter can be used to further expand portions of a stent that has already been expanded into engagement with the main vessel at the vessel bifurcation. The stent is usually positioned in the main vessel spanning the opening into a branch vessel of the vessel bifurcation.

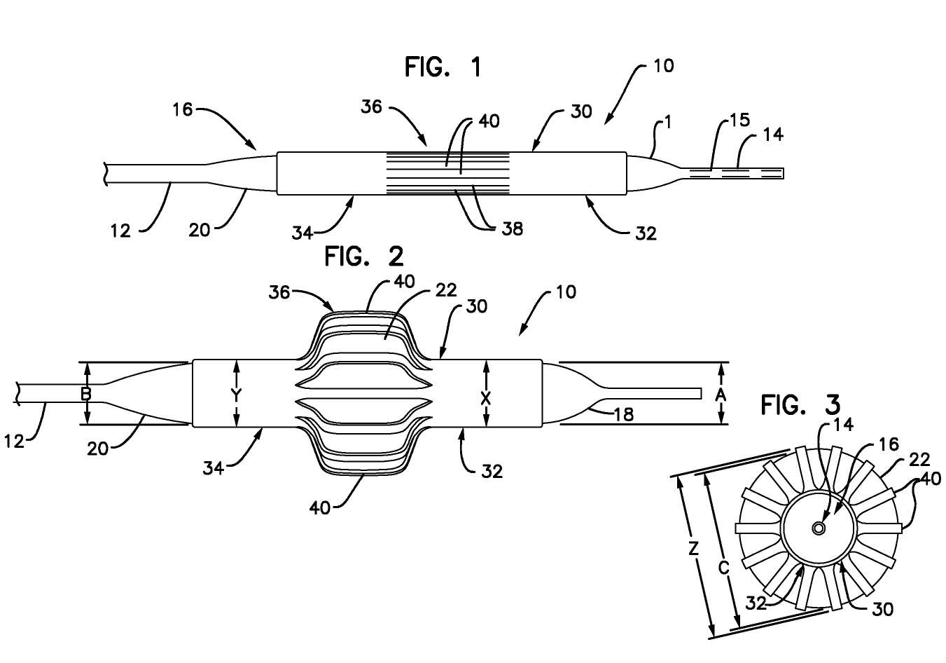

### The Example Catheter Assembly of FIGS. 1-3

An example catheter assembly 10 shown with reference to FIGS. 1-3 includes a shaft 12, a guidewire housing defining a guidewire lumen 15, a balloon member 16, and a balloon restricting arrangement 30. The balloon 16 is positioned at a distal end of the shaft 12. The guidewire housing 14 extends through at least a portion of the shaft 12, through the balloon member 16, and extends distally of the balloon member 16. The balloon member 16 includes a distal portion 18, a proximal portion 20, and a circumferential bulge portion 22. The shape and size of the distal, proximal, and circumferential bulge portions 18, 20, 22 are defined in part as a result of the structure of the balloon restricting arrangement 30. The distal portion 18 has a distal inflation dimension A, the proximal portion 20 has a proximal inflation dimension B, and the circumferential bulge portion has an inflation dimension C when inflated (see FIGS. 2 and 3).

The balloon restricting arrangement 30 includes a distal restricting member 32, a proximal restricting member 34, and a bulge restricting portion 36. The distal, proximal, and bulge restricting members 32, 34, 36 are positioned in axial alignment with the distal, proximal, and circumferential bulge portions 18, 20, 22 of the balloon member 16, respectively. The distal restricting member 32 has a maximum radial dimension X and a proximal edge 44. The proximal restricting member 34 has a maximum radial dimension Y and a distal edge 42. The bulge restricting portion 36 includes a plurality of slits 38 that define a plurality of strips 40. The strips 40 extend from the distal restricting member 32 to the proximal restricting member 34. The strips 40 can be integral with the distal and proximal restricting members 32, 34. Alternatively, the restricting strips 40 can be formed as separate members that are secured to the distal and proximal restricting members 32, 34 in a separate connecting step.

The strips 40 can help maintain the distal and proximal restricting members 32, 34 within a range of axial positions relative to each other. In one example, the distal and proximal restricting members 32, 34 move axially relative to each other as the circumferential bulge portion 22 of the balloon member 16 is inflated, thus decreasing the distance between the distal and proximal restricting members 32, 34. The restricting strips 40 help maintain a connection between the distal and proximal restricting members 32, 34 such that the distal restricting member 34 does not advance distally off of the balloon member 16 and the proximal restricting member 34 does not advance proximally off of the balloon member 16.

The maximum radial dimension X of the distal restricting member 32 and the maximum radial dimension Y of the proximal restricting member 34 is less than a maximum inflated dimension of each of the distal and proximal portions 18, 20, respectively, of the balloon member 16. The maximum inflated dimension of each of the distal and proximal portions 18, 20 when not limited by a restricting structure is greater than radial dimensions X, Y, respectively. The dimensions A, B of the distal and proximal portions 18, 20 of the balloon member 16, when restricted by the distal and proximal restricting members 32, 34 are no greater than dimensions X, Y, respectively. The dimensions A, B, when restricted in size by restricting members 32, 34 can be referred to as being restricted inflated dimensions (e.g., restricted proximal inflated dimension and restricted distal inflated dimension).

The bulge restricting portion 36 can be constructed to provide little to no restriction on the inflation dimension C of the circumferential bulge portion 32 of the balloon member 16. The bulge restricting portion 36 is configured primarily to maintain physical attachment of the distal and proximal restricting members 32, 34 rather than restrict in some way the ability of the circumferential bulge portion 22 to reach its maximum inflated dimension. However, in some arrangements, the radial dimension Z of the bulge restricting portion 36 can be restricted to a size by the bulge restricting portion 36 that is less than a maximum inflated dimension of the circumferential bulge portion 22 that could otherwise occur if the bulge restricting portion were not present.

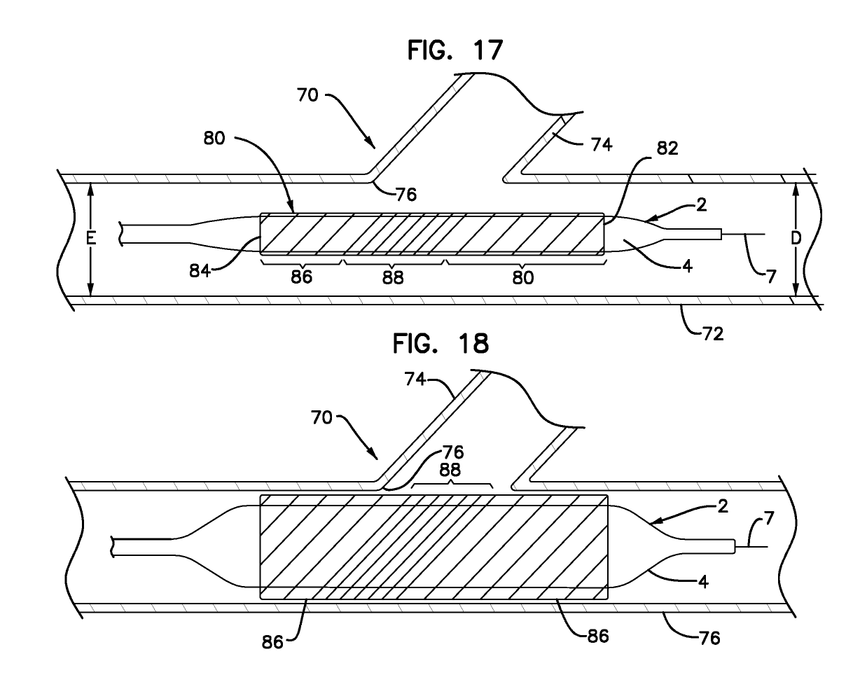

While alternatives are possible, the inflation dimensions A, B are typically less than the inflation dimension C. Likewise, the radial dimensions X, Y are typically less than the dimension Z and the inflation dimension C. Furthermore, the radial dimensions X, Y are typically less than the minimum internal dimension D and E of the vessel bifurcation at locations distal and proximal of the opening into the branch vessel of the vessel bifurcation (see FIG. 17). With the X and Y dimensions typically being less than the dimensions D, E, the inflation of the balloon member 16 does not result in over expansion of the vessels of the vessel bifurcation, overexpansion of a vessel can result in undue stress in the vessel wall that can cause damage to the vessel.

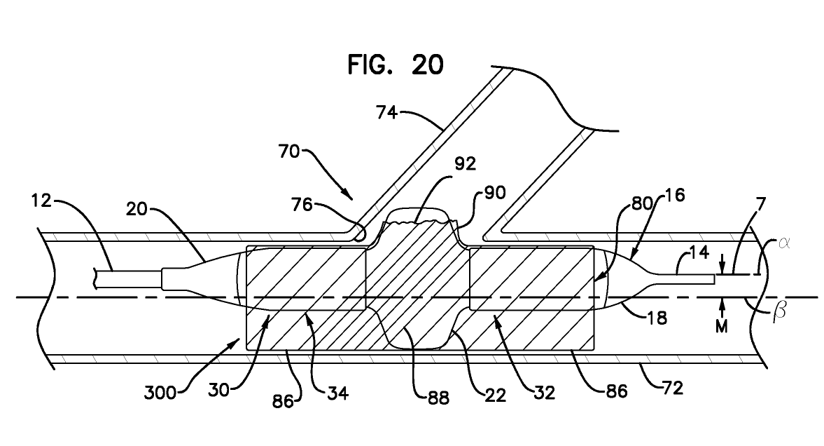

The inflation dimension C and radial dimension Z typically are greater than the vessel dimensions D, E such that a portion of the circumferential bulge portion 22 of the balloon member 16 can extend into the branch vessel while another portion of the circumferential bulge portion 22 engages an opposing side wall of the main vessel of the vessel bifurcation opposite the opening into the branch vessel (i.e., see FIG. 20).

The balloon restricting arrangement 30 limits the expanded size of certain portions of the balloon member (i.e., the distal and/or proximal portion 18, 20) to a given size while permitting a different portion of the balloon member (i.e., the circumferential bulge portion 22) to expand to a different, greater size. The larger inflated sized portion of the balloon member (i.e., the circumferential bulge portion 22) can be used to expand portions of a stent that is located within a main vessel of the vessel bifurcation in a radial outward direction into a branch vessel of the vessel bifurcation.

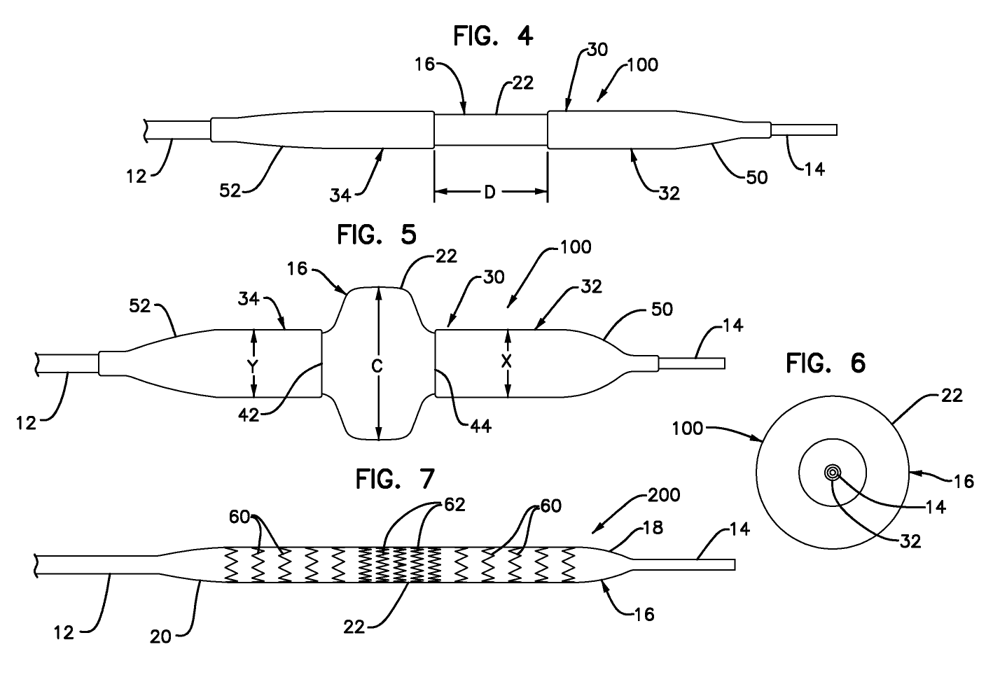

### The Example Catheter Assembly of FIGS. 4-6

Referring now to FIGS. 4-6, another catheter assembly 100 is shown and described. The catheter assembly 100 includes a catheter shaft 12, a guidewire housing 14, a balloon member 16, and a balloon restricting arrangement 30. The balloon member 16 includes a distal portion 18, a proximal portion 20, and a circumferential bulge portion 22. The balloon restricting arrangement 30 includes a distal restricting member 32 and a proximal restricting member 34.

The distal portion 18 of the balloon member 16 is mounted to the guidewire housing 14 at a location distal of the balloon member 16. The proximal portion 20 of the balloon member 16 is mounted to the catheter shaft 12 at a location proximal of the balloon member 16. The catheter shaft 12 defines an inflation lumen therein that is in fluid communication with the balloon member 16.

The distal and proximal restricting members 32, 34 are spaced apart axially from each other a distance D (see FIG. 4). The spacing D can also be referred to an axial gap between a proximal edge 44 of the distal restricting member 32 and a distal edge 42 of the proximal restricting member 34. The distal restricting member 32 has a maximum radial dimension X and the proximal restricting member has a maximum radial dimension Y. The dimensions X and Y can be equal or of different sizes. The radial dimensions X, Y can be configured to restrict the inflated size of the distal and proximal portions 18, 20 of the balloon member 16 to a size less than X and Y, respectively.

The gap or spacing D between the distal and proximal restricting members 32, 34 permits a portion of the balloon member 16 (i.e., circumferential bulge portion 22) to expand to an inflated dimension C that is greater than dimensions X and Y. Typically, the circumferential bulge portion 22 extends radially outward relative to an outer surface of the distal and proximal restricting members 32, 34 the same or similar radial distance at each location around a circumference of the balloon member 16. FIG. 6 illustrates the circumferential bulge portion 22 extending radially outward relative to the outer surface of the distal restricting member 32.

The distal and proximal restricting members 32, 34 can be secured to the distal and proximal portions 18, 20 of the balloon 16 using, for example, laser welding, heat welding, or adhesives. In some arrangements, the distal and proximal restricting members 32, 34 can comprise a semi-compliant material that is expandable to a maximum dimension upon inflation of the balloon member 16, wherein the maximum dimension X, Y is less than the inflation dimension C of the circumferential bulge portion. In other arrangements, the distal and proximal restricting members 32, 34 can comprise a non-compliant material.

The catheter assembly 10 can be constructed of various combinations of materials. For example, the balloon member 16 and balloon restricting arrangement 30 can be constructed as a bi-layer structure wherein the balloon member 16 comprises a compliant or semi-compliant layer of material and the distal and proximal restricting members 32, 34 comprise a non-compliant material layer that covers substantially all of the outer surface of the balloon member 16 except in the area of the circumferential bulge portion 22. The distal restricting member 32 can include a distal tapered portion that extends over a distal waste portion of the balloon 16. The proximal restricting member 34 can include a proximal tapered portion that extends over a proximal waste portion of the balloon 16.

The gap or spacing D between the distal and proximal restricting members 32, 34 permits the semi-compliant or compliant inner layer extend radially outward relative to the outer surface maximum dimension X, Y of the distal and proximal restricting members 32, 34.

### The Example Catheter Assembly of FIGS. 7-10

A further example catheter assembly 200 is described with reference to FIGS. 7-10. The catheter assembly 200 includes a shaft 12, a guidewire housing 14, a balloon member 16, and a balloon restricting arrangement 30. The balloon member 16 includes a distal portion 18, a proximal portion 20, and a circumferential bulge portion 22. The balloon restricting arrangement 30 includes a first set of fiber members 60 positioned in axial alignment with the distal and proximal portions 18, 20 of the balloon member 16, and a second set of fiber members 62 in axial alignment with the circumferential bulge portion 22 of the balloon member 16. The first and second sets of fiber members 60, 62, are positioned on an outer surface of the balloon member 16 and can be connected to the outer surface of the balloon members 16. Alternatively, the first and second sets of fiber members 60, 62 can be embedded within the material defining the balloon member 16. Various fiber embedding techniques can be used to embed the fiber members within the wall structure of the balloon member.

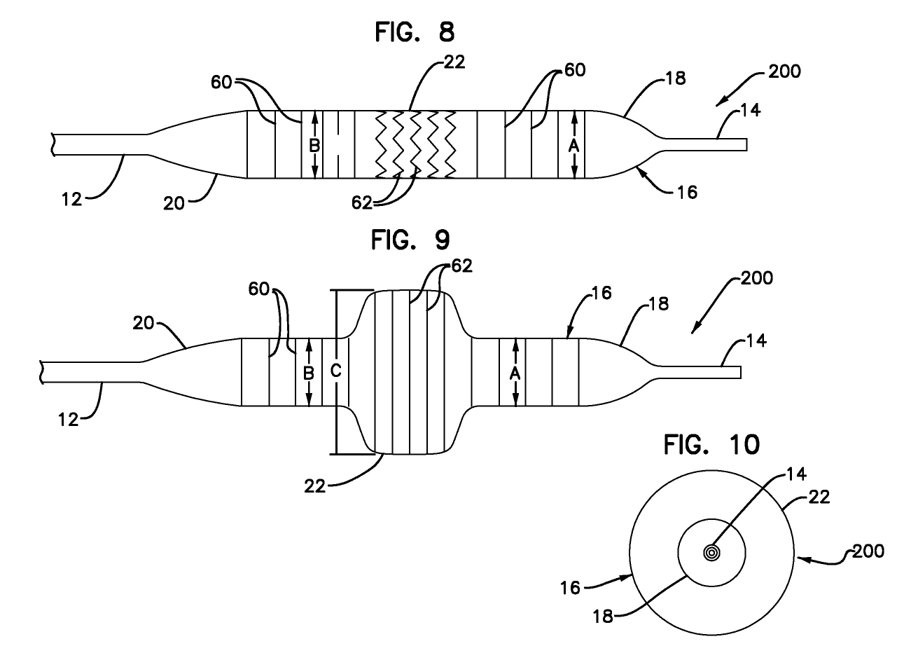

The first and second sets of fiber members 60, 62 typically individually extend around an entire circumference of the balloon member 16 and define a circumferential length. The circumferential length results in a maximum diameter dimension. The maximum diameter dimension of the first set of fiber members 60 result in a maximum inflated dimension A, B of the distal and proximal portions 18, 20 of the balloon member 16, respectively. FIG. 8 illustrates the catheter assembly 10 with the balloon 16 inflated to a state where the first set of fiber members 60 are extended resulting in the maximum diameter dimension A, B of the balloon 16. Further inflation of the balloon member 16 results in further inflation of the circumferential bulge portion 22 of the balloon member 16 as the second set of fiber members 60 are elongated into a maximum diameter dimension C. The circumferential length of the second set of fiber members 62 is greater than the circumferential length of the first set of fiber members 60, thus resulting in a maximum diameter dimension C for the circumferential bulge portion 22 that is greater than the maximum dimensions A, B of the distal and proximal portions 18, 20.

Typically, the first and second sets of fiber members 60, 62 have elastic properties such that when the balloon member 16 is deflated the fiber members 60, 62 can return to a compressed state as shown in FIG. 7 as compared to the expanded, elongated state shown in FIG. 9.

The material of the first and second sets of fiber members 60, 62 can be semi-compliant or non-compliant material. The fiber members 60, 62 can have a generally circular cross-section or can have other cross-sectional shapes such as rectangular or generally flat, ribbon-shaped construction. In some arrangements, the fiber members can be imbedded in or carried by a sleeve member that is positioned on the balloon member 16. Such a sleeve member can comprise compliant or semi-compliant material and the fiber members can be secured to an outer or inner surface of the sleeve member or imbedded in the sleeve member.

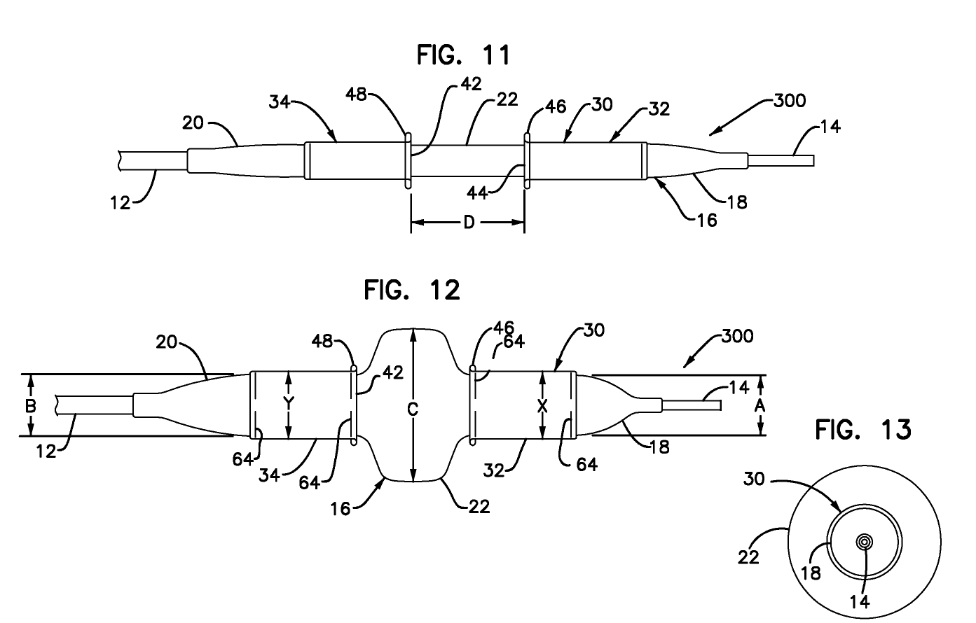

### The Example Catheter Assembly of FIGS. 11-13

Another example catheter assembly 300 is shown and described with reference to FIGS. 10-13. The catheter assembly 300 includes a shaft 12, a guidewire housing 14, a balloon member 16, and a balloon restricting arrangement 30. The balloon member 16 includes a distal portion 18, a proximal portion 20, and a circumferential bulge portion 22. The balloon restricting arrangement 30 includes a distal restricting member 32 and a proximal restricting member 34. The distal and proximal restricting members 32, 34 are spaced apart axially a distance D that defines an axial gap between a proximal edge 44 of the distal restricting member 32 and a distal edge 42 of the proximal restricting member 34 (see FIG. 11).

The distal and proximal restricting members 32, 34 have a length measured in the axial or longitudinal direction that does not extend distally or proximally over the tapered distal and proximal end of the balloon member 16. The distal and proximal restricting members 32, 34 can comprise a semi-compliant or non-compliant material that has a maximum expanded size X, Y, respectively, that is less than a maximum possible inflated size of the portions 18, 20 of the balloon member 16. The gap D between the distal and proximal restricting members 32, 34 permits that portion of the balloon member 16 in the area of the circumferential bulge portion 22 to inflate to a maximum inflation dimension C that is greater than the dimensions X, Y. The resulting circumferential bulge portion 22 of the balloon 16 can be used to expand a portion of a stent from a main vessel into a branch vessel at a vessel bifurcation as will be described in further detail below.

The distal and proximal restricting members 32, 34, can be secured to the balloon member 16 using a plurality of connection points 64. The connection points 64 can be formed using, for example, heat welding, laser welding or adhesives. The distal restricting member 32 can include a rolled proximal edge 46 and the proximal restricting member can include a rolled distal edge 48. The rolled edges 46, 48 can provide further limits to expansion of the restricting members 32, 34 adjacent to the circumferential bulge portion 22 to a size greater than dimensions X, Y. Further, the rolled edges 46, 48 can provide a more contoured interface between the restricting members 32, 34 and the circumferential bulge portion 22 that can reduce incidence of damage to the circumferential bulge portion 22.

Any of the balloon restricting arrangements 30 described with reference to FIGS. 1-13 can comprise a plurality of cuts or grooves 54 defined therein as shown in the distal restricting member 32 of FIG. 14. The cuts or grooves 54 can be arranged at a diagonal so as to be continuous feature extending along the length of that portion of the balloon restricting arrangement. The cut or groove 54 can provide additional flexibility in that portion of the balloon restricting arrangement 34 for the purpose of, for example, improved ease in navigating the catheter assembly through tortuous shapes within a vessel.

The balloon restricting arrangements described herein can also include one or more marker members 66. The marker members 66 can be viewable from outside of a patient using, for example, fluoroscopy and X-ray technology. The marker members 66 can be positioned, for example, on a distal restricting member 32 near the proximal edge 44 thereof to help the operator visualize features of the catheter assembly at or near the circumferential bulge portion 22. As described above, positioning the catheter assembly 10 with the circumferential bulge portion 22 in axial arrangement with an opening into a branch vessel at a vessel bifurcation is important in the method of expanding a portion of a stent from within the main vessel into a branch vessel of the vessel bifurcation.

In some arrangements, the marker member 66 extends along an entire length of at least a portion of the balloon restricting arrangement (e.g., along at least one of the distal or proximal restriction members 32, 34). In other arrangements, multiple markers 66 can be used at various locations on the balloon restricting arrangement. In some arrangements, the marker extends around an entire circumference of at least a portion of the balloon restricting arrangement, while in other arrangements the marker extends around only a portion of a circumference of the balloon restricting arrangement.

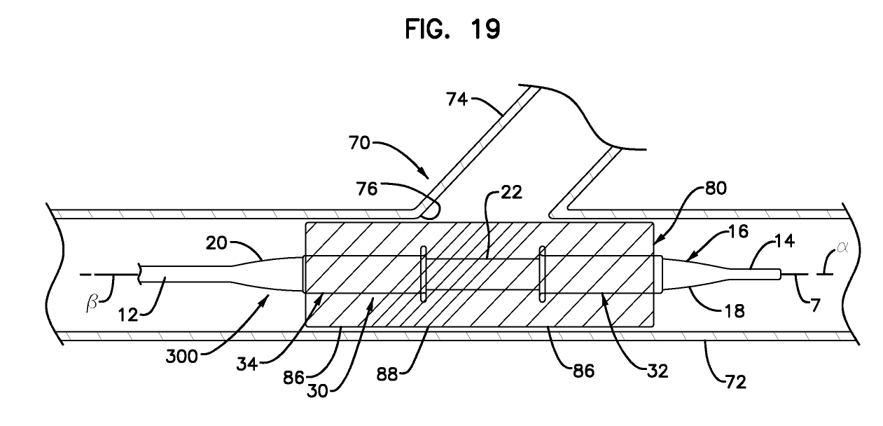

### The Example Treatment Method of FIGS. 17-21

An example method of treating a vessel bifurcation 70 is now shown and described with reference to FIGS. 17-21 using catheter assembly 300 described above and a stent having a stent construction similar to the stent 80 shown in FIG. 22.

Referring first to FIG. 17, although many alternative methods are possible, this example method begins by advancing a guidewire 7 to a vessel bifurcation 70 to a position within a main vessel 72 at a location distal of an opening or ostium 76 into a branch vessel 74. A stent positioning catheter 2 carrying a stent 80 is advanced over the guidewire 7 to the vessel bifurcation 70. The stent 80 includes a distal open end 82, a proximal open end 84, a low density strut arrangement 86, and a high density strut arrangement 88 (see FIG. 22). The stent positioning catheter 2 is adjusted in the axial direction until the high density strut arrangement 88 is positioned in axial alignment with the opening 76 into the branch vessels 74.

Some example constructions for the stent 80 are disclosed in U.S. Patent Publication No. 2007/0233270, and U.S. Patent Publication No. 2007/0239257, which are incorporated herein by reference.

The low density strut arrangement 86 can include a plurality of struts that are spaced apart axially further than the axial spacing of the high density structure arrangement 88. The high density strut arrangement 88 can include at least one strut member that has a length when the strut member is in a fully expanded state that is longer than fully extended lengths of the strut members of the low density strut arrangement 86. The high density strut arrangement 88 can have fewer connecting points between adjacent struts as compared to the number of connecting points between struts of the low density strut arrangement 86. Reducing the connecting points between adjacent struts can help create a larger side opening in the stent, such as a side opening that provides access from the main vessel into the branch vessel at a vessel bifurcation.

A balloon member 4 of the stent positioning catheter 2 is then inflated to expand the stent 80 into engagement with the vessel wall of the main vessel 76 (see FIG. 18). The balloon member 4 is then deflated and the stent positioning catheter 2 is retracted proximally along the guidewire 7 out of the patient. The catheter assembly 300 is then advanced over the guidewire 7 to the vessel bifurcation 70 until the circumferential bulge portion 22 of the balloon member 16 is arranged in axial alignment with the opening 76 into the branch vessel 74. A marker or other feature of the catheter assembly 300 can be used to help the operator visually understand the relative position between the circumferential bulge portion 22 and the opening 76 near the branch vessel 74.

In one example, a marker is positioned at a distal end portion of the catheter assembly 300, such as at a distal end, a proximal end or a central portion of the balloon member 16. In an alternative arrangement, each of the stent positioning catheter 2 and the catheter assembly 300 can include a marker positioned at a proximal end thereof that remains outside the patient. In such an arrangement, the marker is positioned on the catheter assembly 300 (e.g., on a guidewire housing thereof) a distance from the bulge portion 22 that is equal to a distance from the marker positioned on the stent positioning catheter 2 (e.g., on a guidewire housing thereof) to a feature of the stent 80 (such as a center point along a length of the stent 80) or balloon member 4 that is to be located at the vessel bifurcation. Typically, a path of each of catheter 2 and assembly 300 to the vessel bifurcation is the same distance, so positioning the marker of each at the same relative location outside of the patient would position the bulge portion at the opening 76 of the vessel bifurcation.

The balloon member 16 is then inflated as shown in FIG. 20. The inflated circumferential bulge portion 22 engages against an interior of the stent 80 at a location on the main vessel wall opposite the opening 76 into the branch vessel 74, thereby shifting the catheter assembly 300 radially away from a center line β of the main vessel 72 to a position where a central axis α of the catheter assembly 300 is spaced a distance M from axis β (see FIG. 20). The maximum radial size of the distal and proximal restricting members 32, 34 is less than the internal minimum dimensions D, E of the vessel 72 so that no further expansion of the stent in the area distal and proximal of the opening 76 and the branch vessel 74 occurs, which further expansion might damage or cause stress to the main vessel wall.

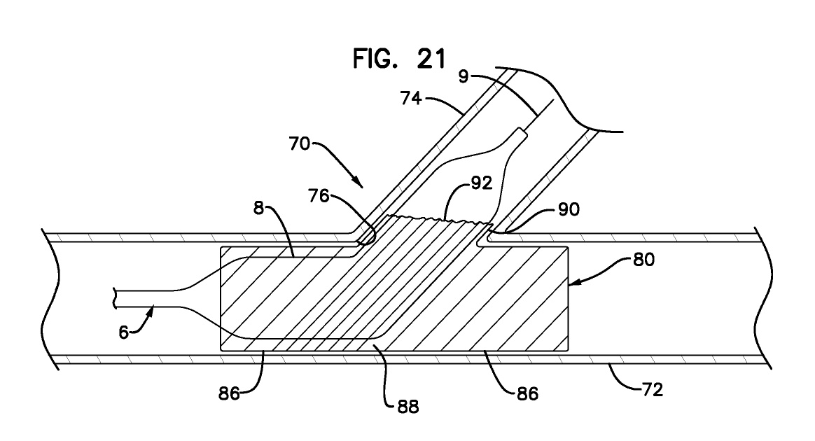

The circumferential bulge portion 22 in the inflated state shown in FIG. 20 has a portion thereof that engages the high density strut arrangement 88 of stent 80 to move a portion of the stent 80 in a radial outward direction into the branch vessel 74. FIG. 20 illustrates a portion 90 of the stent 80 that extends in a radial outward direction through the opening 76 into the branch vessel 74. As this portion 90 extends in the radial outward direction, a side opening 92 can be defined in the stent between adjacent strut members that have been extended into the branch vessel. The side opening 92 can be used as an opening through which additional devices can be advanced for further treatment of the branch vessel 74 as shown in FIG. 21. Further, opening of the side opening 92 by moving the struts into engagement with the branch vessel wall can provide a less obstructed pathway for blood flow to move from the main vessel 72 into the branch vessel 74.

The balloon member 16 can then be deflated and the catheter assembly can be removed proximally from the patient. In a further treatment step, a guidewire 9 can be advanced into the branch vessel 74 and a post dilation balloon catheter 6 can be advanced over the guidewire 9 and extend through the side opening 92 in the stent 80. A balloon member 8 of the balloon catheter 6 can be inflated to further expand the side opening 82 and move the portion 90 into further engagement with the branch vessel 74 in the area of the opening 76. In a still further treatment step, a secondary stent can be advanced through the side opening 92 and into the branch vessel 74 with a portion of a secondary stent overlapping with the portion 90 of stent 80. The secondary stent can be expanded into engagement with the portion 90 and other portions of the branch vessel 74 for treatment of the vessel bifurcation 70.

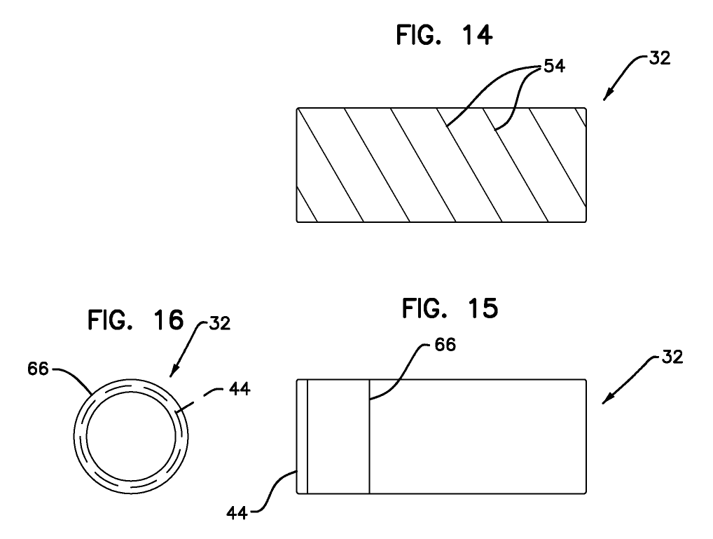

Many other treatment methods including additional or varied steps from those described with reference to FIGS. 17-21 can be used with aspects of the catheter assemblies described above with reference to the attached figures. Furthermore, many different balloon restricting arrangements are possible that together with a balloon member can result in creation of a circumferential bulge portion in the balloon member that is used to expand a portion of an already expanded stent in a post-dilation procedure such as the method described above with reference to FIGS. 17-21. Any of the features described with reference to FIGS. 1-21 can be combined in any desired combination to provide alternative arrangements and treatment methods within the scope of the present disclosure.

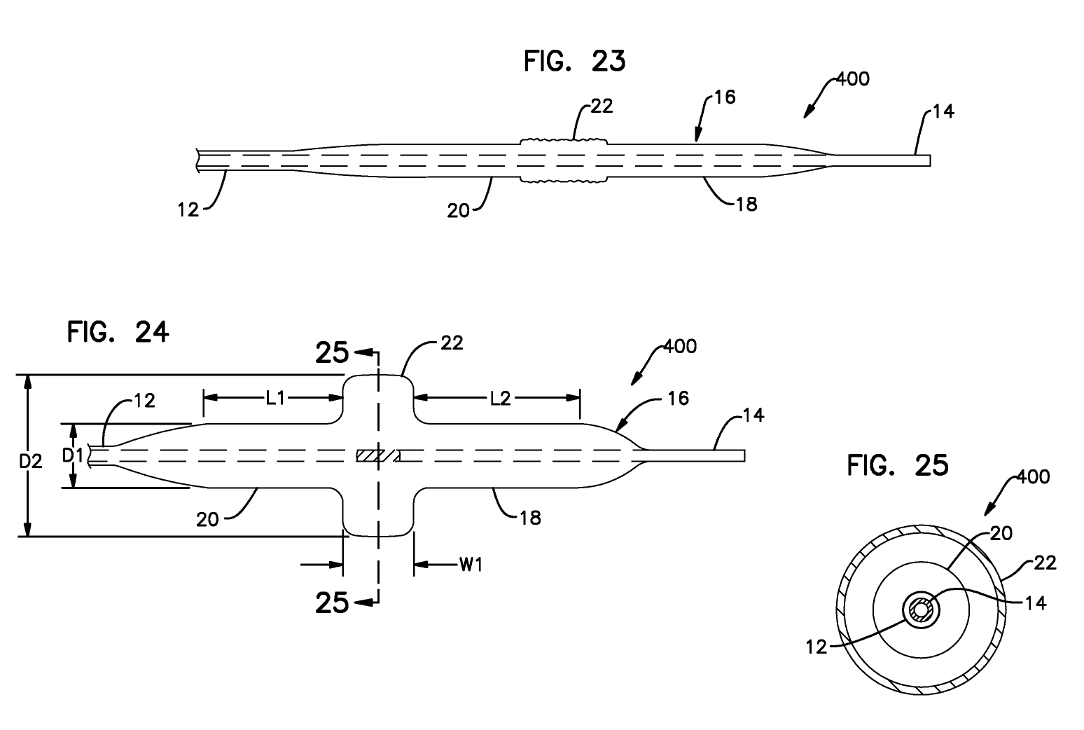

### The Example Catheter Assembly of FIGS. 23-25

Referring now to FIGS. 23-25, an example catheter assembly 400 is shown and described. Catheter assembly includes a shaft 12, a guidewire housing 14, and a balloon member 16. The balloon member 16 includes a distal portion 18, a proximal portion 20, and a circumferential bulge portion 22. The proximal end portion 20 is positioned proximal of the circumferential bulge portion 22, and the distal end portion 18 is positioned distal of the circumferential bulge portion 22. The distal and proximal portions 18, 20 together can define a main body portion of the balloon member about which the circumferential bulge portion 22 extends.

FIG. 23 illustrates the balloon member 16 in a deflated state. FIGS. 2 and 3 illustrate the balloon member 16 in an inflated state. The distal and proximal end portions 18, of the inflated balloon member define a maximum balloon dimension D1. The dimension D1 can be different for each of the distal and proximal portions 18, 20. The portions 18, 20 can also have lengths L1, L2, respectively, that are measured relative to the circumferential bulge portion 22. The lengths L1, L2 can be the same or different in alternative arrangements. The inflated circumferential bulge portion 22 has a maximum inflated dimension D2, wherein the dimension D2 is greater than the dimension D1. The circumferential bulge portion 22 also has a width W1 defined in an axial direction along the balloon member 16.

The cross-sectional view of FIG. 25 shows the circumferential bulge portion 22 having a relatively constant dimension D2 at each location around the circumference of the circumferential bulge portion 22. As will be described in further detail below, the circumferential bulge portion 22 can be used to treat a vessel bifurcation by extending from within the main vessel of the vessel bifurcation radially outward through the branch vessel ostium regardless of the radial orientation of the circumferential bulge portion 22 relative to the branch vessel.

In one example, the dimension D1 is in the range of about 1 to about 3 mm and more preferably about 1.5 to about 2.5 mm. The dimension D2 can be in the range of about 2 to about 6 mm, and more preferably about 3 to about 5 mm. The dimension D2 can also be defined in relationship to the size of D1. For example, D2 can be in the range of about 25% to about 200% greater than D1, and more preferably about 50% to about 150% the size of D1.

The width W1 is typically in the range of about 0.5 to about 4 mm, and more preferably about 1 to about 1.5 mm. The dimensions D1, D2, W can vary depending on, for example, the size of the vessels being treated at the vessel bifurcation including the size of the ostium into the branch vessel. Typically, each of the lengths L1, L2 of portions 40, 42, respectively, is between about 2 mm and about 10 mm. The value of L1 and L2 can vary relative to each other.

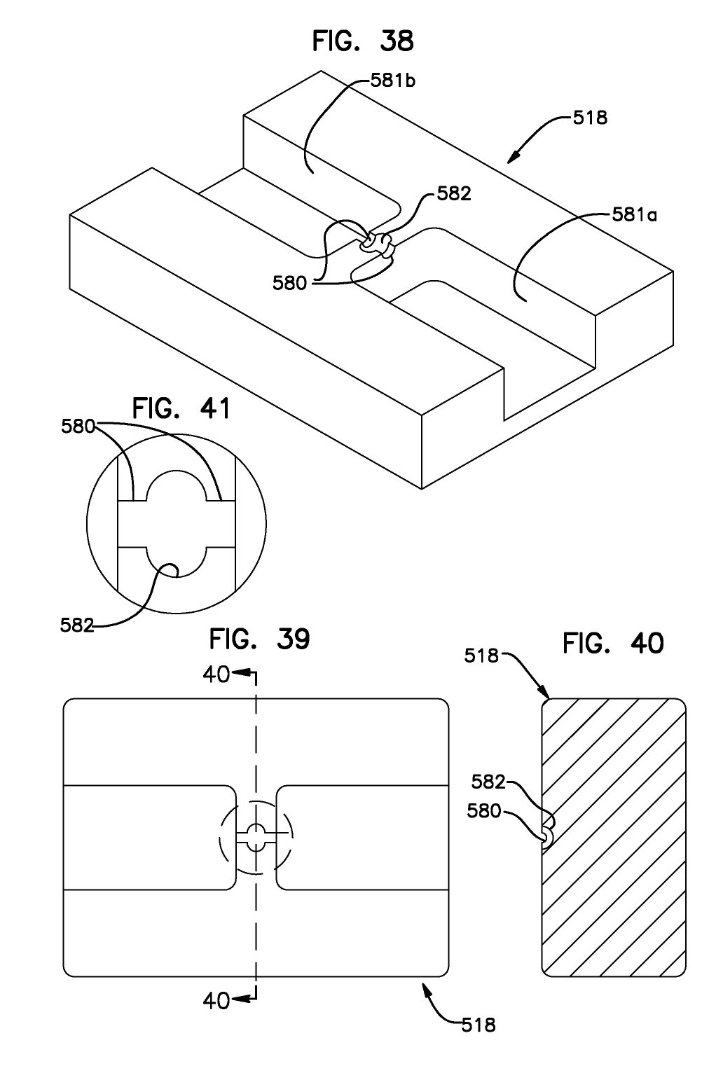

The circumferential bulge portion 22 can be formed in the balloon member 16 using a molding process. FIGS. 38-41 illustrate an example portion of a balloon mold that could be used to form the circumferential bulge portion 22 in the balloon member 16. The balloon mold body 518 includes a main balloon cavity portion 580 sized to receive a length of hollow cylindrical catheter material. A bulge portion cavity 582 is defined in the balloon mold body 518. A pair of mold inserts (not shown) can be positioned within mold insert recesses 581A, B on opposing sides of the bulge portion cavity 582. Each of the mold inserts (not shown) can define an additional length of balloon cavity aligned with the main balloon cavity portion 580.

Commercial high strength balloons having wall strengths in excess of 20,000 psi, have been formed of a wide variety of polymeric materials, including PET, nylons, polyurethanes and various block copolymer thermoplastic elastomers. U.S. Pat. Nos. 4,490,421 and 5,264,260 describe PET balloons. U.S. Pat. Nos. 4,906,244 and 5,328,468 describe polyamide balloons. U.S. Pat. Nos. 4,950,239 and 5,500,180 describe balloons made from polyurethane block copolymers. U.S. Pat. Nos. 5,556,383 and 6,146,356 describe balloons made from polyether-block-amide copolymers and polyester-block-ether copolymers. U.S. Pat. No. 6,270,522 describes balloons made from polyester-block-ether copolymers of high flexural modulus. U.S. Pat. No. 5,344,400 describes balloons made from polyarylene sulfide. All of these balloons are produced from extruded tubing of the polymeric material by a blow-forming radial expansion process. U.S. Pat. Nos. 5,250,069; 5,797,877; and 5,270,086 describe still further materials which may be used to make such balloons. A further list of balloon and catheter shaft materials is provided below.

An example method of producing a balloon such as the balloon member 16 includes application of heat, internal pressure, and axial tensioning on a length of hollow cylindrical catheter material captured within the main balloon cavity portion 580 and bulge portion cavity 582. The resulting structure of this method provides the distal and proximal portions 18, 20 and circumferential bulge portion 38 in the balloon member 16.

An alternate method can include, after an initial mold process of forming the main balloon portion 16 (i.e., a main balloon portion having a maximum dimension D1 of about 4 to 5 mm), creating in a secondary step the circumferential bulge portion 22 in a secondary molding process. The secondary molding process can include selectively heating the balloon material of the main balloon portions 18, 20 and shrinking or necking the balloon material down to a desired maximum dimension (e.g., about 2 to 3 mm) on the proximal or distal ends portions 18, 20 of the balloons to create the circumferential bulge portion. This secondary process can include heating the balloon material to a certain temperature that makes the balloon material soft enough so for the balloon material to shrink down. This secondary process can be done with direct contact of heated elements such as stainless steel or Teflon. Alternatively, the secondary process can be accomplished by applying heated air to the balloon material allowing the balloon material to recover from the initial molding expansion and causing the maximum dimension to decrease.

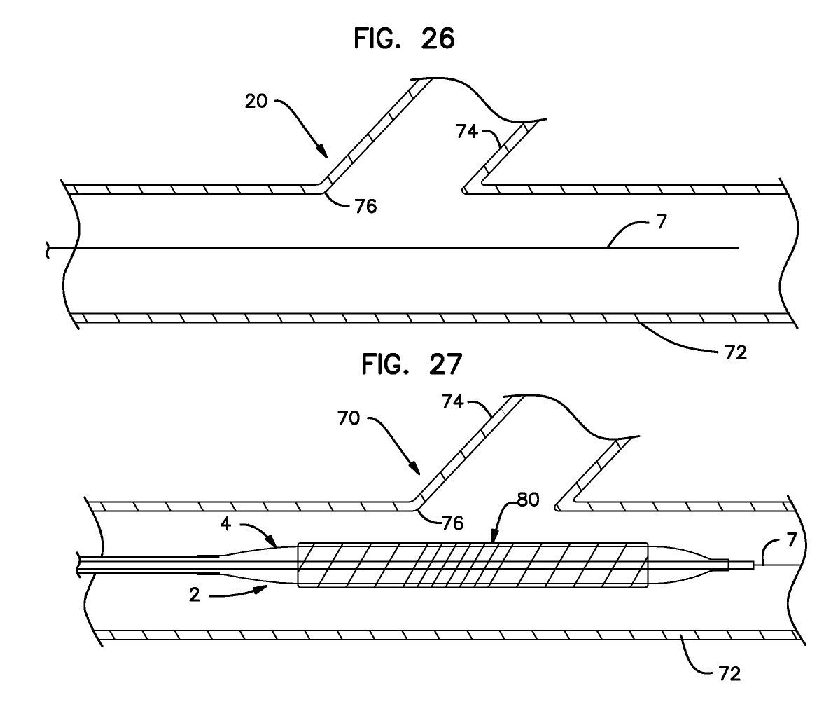

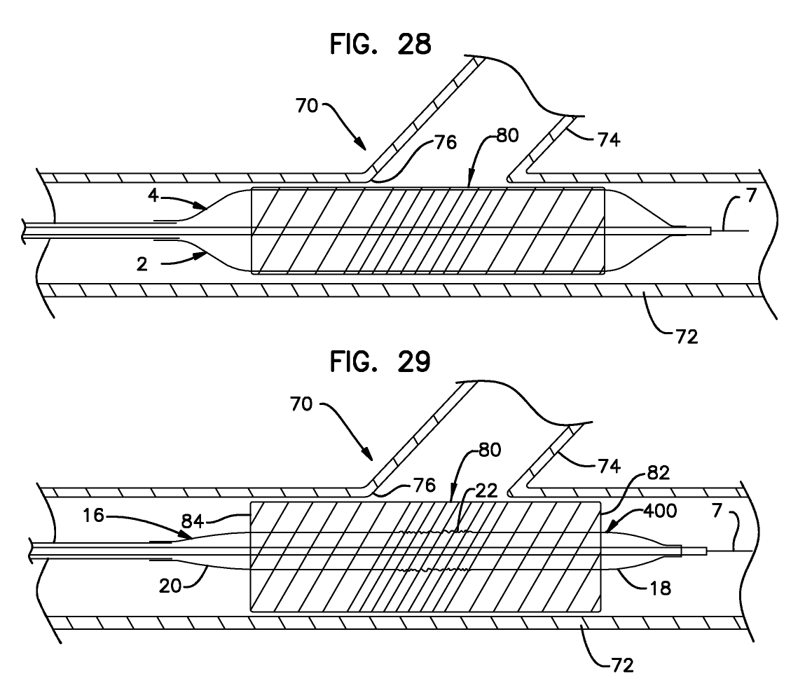

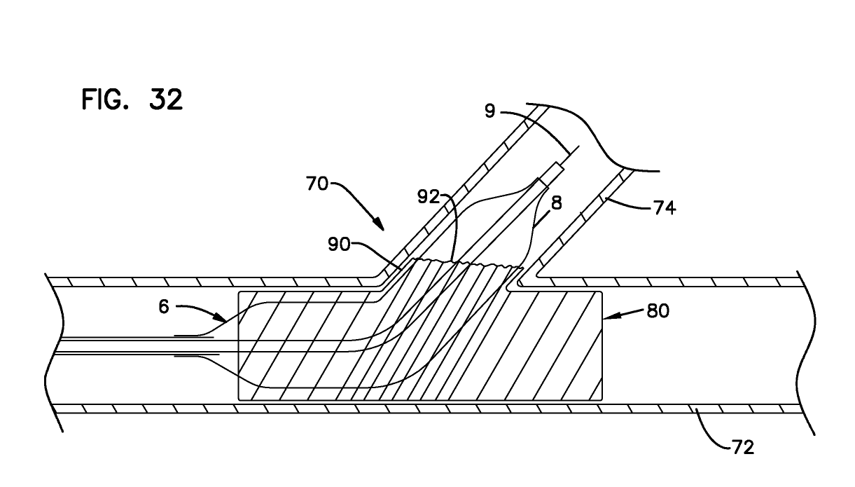

### The Treatment Method of FIGS. 26-32

FIGS. 26-32 illustrate an example method of treating a vessel bifurcation 70. The vessel bifurcation 70 includes a main vessel 72, a branch vessel 74, and ostium 76 defined as an opening from the main 72, 90 into the branch vessel 74.

Initially, a guidewire 7 is advanced through the main vessel 72 to a location distal of the ostium 76. A stent positioning catheter 2 is then advanced over the guidewire 7 to a location spanning the ostium 76 of the branch vessel 74. The stent positioning catheter 2 includes a constant diameter balloon 4 upon which a stent 80 is positioned. Typically, the stent 80 is mounted to the balloon 4 using, for example, crimping or other way of releaseably mounting the stent 80 to the balloon 4. The balloon 4 is then inflated to expand the stent 80 into engagement with the main vessel 72 on a side opposing the ostium 76 into the branch vessel 74.

Referring now to FIG. 29, the stent positioning catheter 2 is retracted proximally over the guidewire 7 and the catheter assembly 400 is advanced over the guidewire 7 for further treatment of the vessel bifurcation 70. The catheter assembly 400 includes a balloon member 16 having a circumferential bulge portion 22. The catheter assembly 400 can be replaced with any of the catheter assemblies 10, 100, 200, 300 and variations thereof described above that define a circumferential bulge portion for expansion of a portion of the stent 80 into the branch vessel 74. The catheter assembly 400 is advanced to an axial position wherein the circumferential bulge portion 22 is aligned with the ostium 76 of the branch vessel 74. In some arrangements, the balloon 16 is structured such that the proximal end portion 20 is positioned proximal of a proximal open end 84 of the stent 80, and the distal end portion 18 is positioned distal of the distal open end 82 of the stent 80.

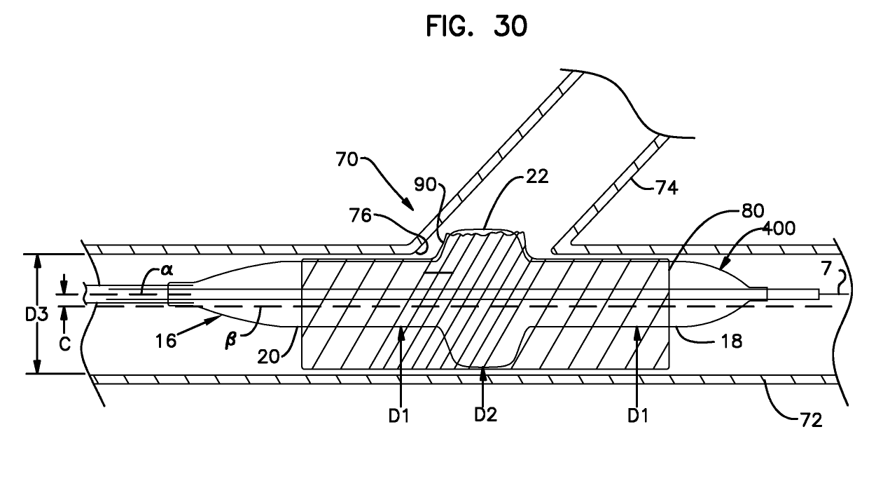

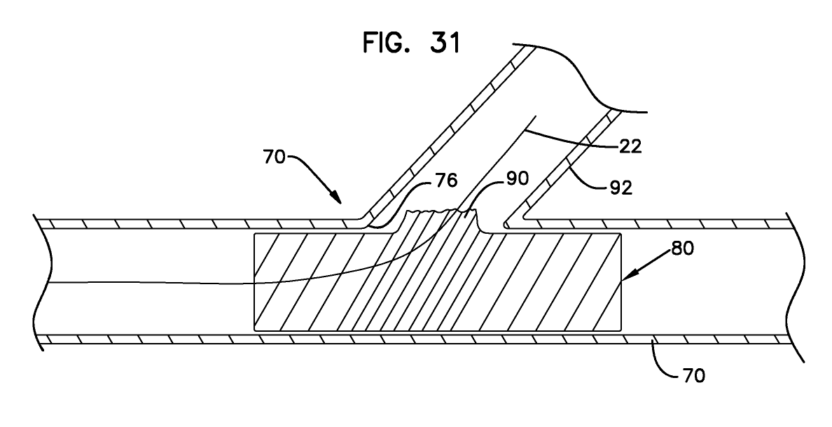

Referring now to FIG. 30, the balloon member 16 is inflated, wherein the circumferential bulge portion 22 radially expands the stent 80 radially outward into the branch vessel 76. As discussed above with reference to FIGS. 30 and 31, inflation of the circumferential bulge portion 22 of the balloon 16 causes not only radially outward expansion of strut members of the stent 80, but also provides an increased spacing between struts in the area of the ostium 76 into the branch vessel 74. Increased spacing between the stent struts in the area of the ostium can provide easier navigation of a guidewire and other treatment devices through the stent sidewall and into the branch vessel 74.

The dimension D1 of the distal and proximal portions 18, 20 of the balloon member 16 is sized smaller than a maximum internal dimension D3 of the main vessel 72. The dimension D2 of the circumferential bulge portion 22 is sized greater than the internal dimension D3 of the main vessel 72. As the balloon member 16 inflates, the catheter assembly 400 shifts in the radial direction towards the branch vessel 74 so that an axis α of the main catheter branch 12 is offset from a central axis β of the main vessel 72 a distance C. Shifting of the main catheter branch 12 within the main vessel 72 a distance C provides for expansion of the stent 80 into the branch vessel 74 while limiting the amount of stress applied by the circumferential bulge portion 22 on a portion 91 of the main vessel 72 opposite the ostium 76.

In arrangements where the dimension D1 is substantially equal to the internal dimension D3 of the main vessel 72 and the circumferential bulge portion dimension D2 is greater than the dimensions D1, D3, the circumferential bulge portion 22 can apply undesired forces and stress upon the area 91 of the main vessel 72 in addition to undesired expansion of the stent 80 in areas other than the ostium 76 of the branch vessel 74.

After expansion of the stent 80 radially outward into the branch vessel 74, which also provide spacing apart of the plurality of rows of struts in the area of the ostium 76 of the branch vessel 74, the balloon member 16 is deflated and retracted from the vessel bifurcation 70. Referring now to FIG. 31, a guidewire 9 can be advanced through the expandable structure 90 and a side opening 92 and into the branch vessel 74. With the guidewire 9 positioned within the branch vessel 74, a further treatment device, such as a post dilatation balloon catheter 6 can be advanced along the guidewire 9, through the expandable structure 90, and at least partially extending into the branch vessel 74. Inflation of a balloon member 8 of the post dilatation balloon catheter 6 can further extend and expand the expandable structure 90 and enlarge the side opening 92 in the stent 80. Typically, it is desirable to expand the expandable structure 90 sufficiently to provide engagement of the stent 80 with the branch vessel 74.

Further treatment of the vessel bifurcation 70 can include deploying a branch stent within the branch vessel 74 that is advanced through a side opening 92 in the expandable structure 90 and at least partially overlaps the expandable structure 90.

The use of a circumferential bulge portion 22 can be particularly important for moving the struts of the stent 80 from the main vessel 72 into the branch vessel 74. The post dilatation balloon catheter 6 typically is most effective in pushing the expandable structure 90 of the stent 80 towards the wall of the branch vessel 74 after the expandable structure 90 has already been at least partially extended through the ostium 76 (see FIG. 22). Usually the farther the circumferential bulge portion 22 can move the expandable structure 90 into the branch vessel 72, the more effective the catheter assembly 400 can be in helping treat the vessel bifurcation 70.

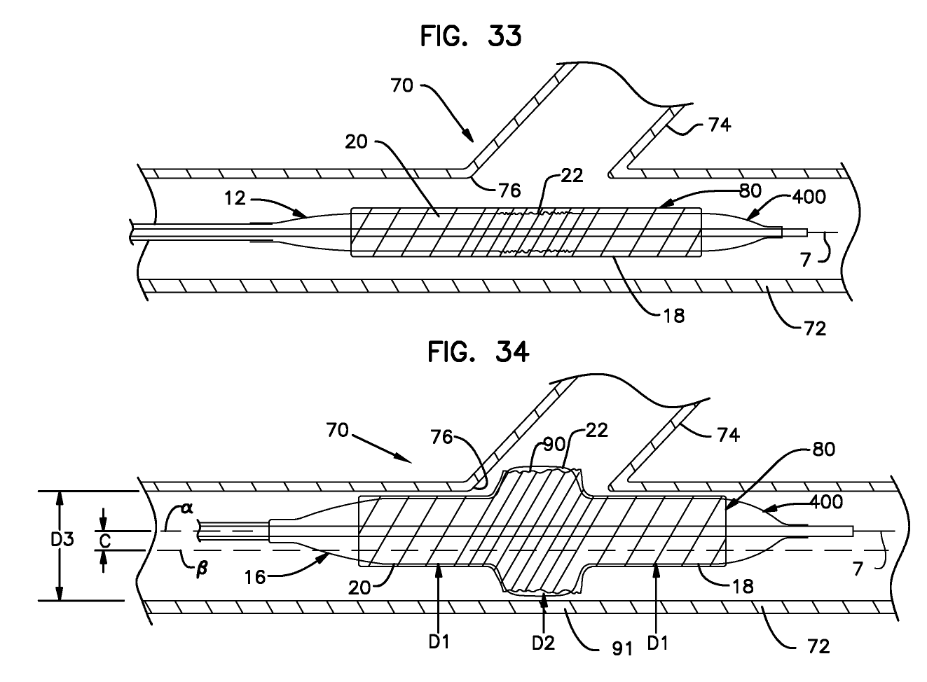

### The Treatment Methods of FIGS. 33-37

FIGS. 23-30 illustrate method steps for at least partially deploying a stent 80 at a vessel bifurcation 70 using the catheter assembly 400 described above. While the illustrated example includes the use of catheter assembly 400 having a balloon member 16 with a circumferential bulge portion 22, other catheter assemblies that include alternative arrangements to provide a circumferential bulge portion, such as those described with reference to FIGS. 1-22 can also be used in these methods of treating vessel bifurcation 70.

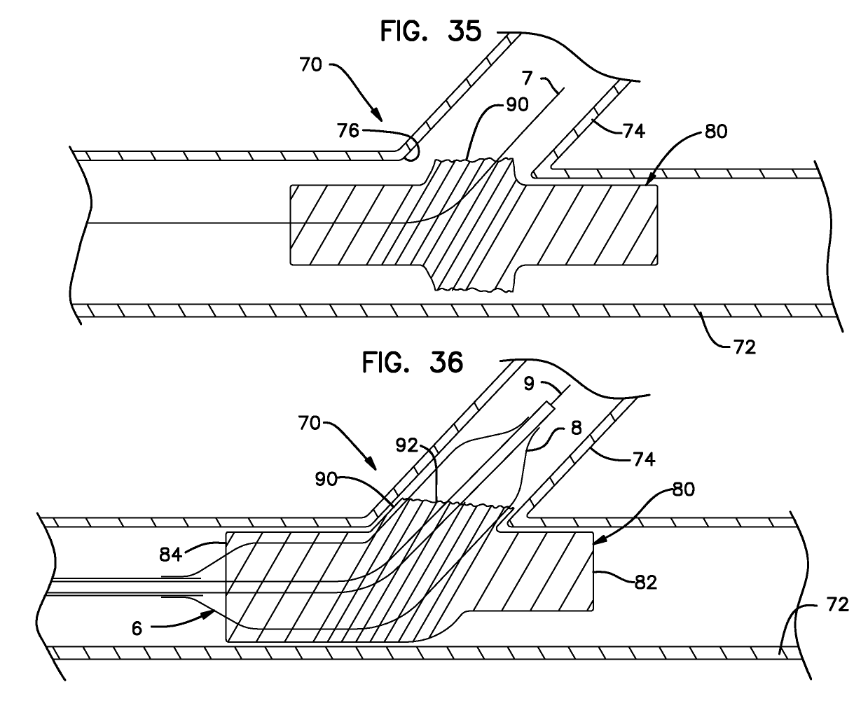

FIGS. 33-34 illustrate initial steps in the treatment methods described further with reference to FIGS. 35-37. A first step of the method includes advancing a guidewire 7 within the main vessel 72 to a location spanning across the ostium 76 of the branch vessel 74 (e.g., see FIG. 33). The catheter assembly 400 is then advanced over the guidewire 7 to a position in which the circumferential bulge portion 22 of the balloon member 16 is axially aligned with the ostium 76. The balloon member 16 can be arranged at any radially rotated position relative to the ostium 76 due to the relatively constant shape and size of the bulge portion 22 around a circumference of the balloon member 16. Referring to FIG. 34, the balloon member 16 is then inflated to at least partially expand the distal and proximal portions 18, 20 of the balloon member 16 and the expandable structure 90.

Typically, the outer dimension D1 of the distal and proximal portions 18, 20 is less than the internal dimension D3 of the main vessel 72. The dimension D2 of the circumferential bulge portion 22 is typically greater than the dimension D3. Inflating the balloon member 16 tends to shift the catheter assembly 400 radially towards the branch vessel 74 a distance C defined between an axis α of the catheter assembly 400 and a central axis β of the main vessel 72. The difference in dimensions D1, D3 typically results in limited engagement between the stent 80 and the main vessel 72 except in the area of expandable structure 90 that is expanded by the circumferential bulge portion 22. Typically, inflation of the circumferential bulge portion 22 provides expansion of the expandable structure 90 sufficient to create an engagement with the main vessel 72 that secures the stent 80 in a radial and axial position relative to the ostium 76 without creating undue stress in the area 91 of the main vessel 72 opposite the ostium 76. After desired expansion of expandable structure 90 of the stent 80, the balloon member 16 can be deflated and the catheter assembly 400 retracted proximally from the patient.

Further treatment of the vessel bifurcation 70 can be performed in different ways as described now with reference to FIGS. 35-37. Referring now to FIGS. 35-37, after retracting the catheter assembly 400, a guidewire 9 can be advanced through a side wall of the stent 80 at the area of the expandable structure 90 and into the branch vessel 74. The use of some stent constructions, such as the example stent described with reference to FIG. 22, can provide improved spacing between adjacent struts in the expandable structure 90 that provides improved ease in advancing the guidewire 9 through the stent sidewall and into the branch vessel 74.

Referring to FIG. 36, a post dilatation balloon catheter 6 is advanced along the guidewire 9, through the expandable structure 90, and at least partially extending into the branch vessel 74. Inflation of a balloon member 8 of the catheter 6 creates an expanded side opening 92 in the expandable structure 90. Expansion of the balloon member 8 can also provide engagement of the stent struts in the expandable structure 90 into engagement with portions of the branch vessel 74. Expansion of balloon member 8 within that portion of the stent 80 proximal of the expandable structure 90 can further define the side opening 92. Expansion of balloon member 8 can also expand that portion of the stent 80 proximal of the expandable stent structure 90 into engagement with the main vessel 72 as shown in FIG. 36. That portion of the stent 80 distal of the expandable structure 90 can still remain in a partially expanded state that is not fully engaged with the main vessel 72.

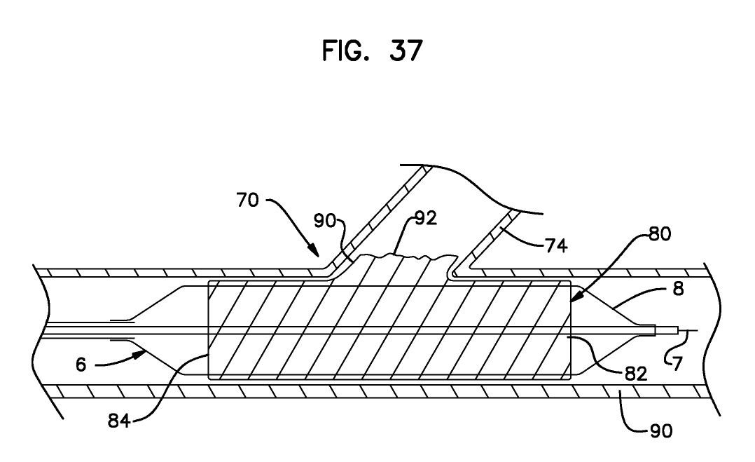

After inflation of the balloon member 8 within the branch vessel 74, the balloon 8 can be at least partially deflated and retracted proximally to a position proximal of the expandable structure 90. The guidewire 9 can also be retracted proximally out of the branch vessel 74 and side opening 92. Either the guidewire 9 or the guidewire 7 can be advanced distally in the main vessel 72 through the distal open end 82 of the stent 80. The post dilatation catheter 6 (or an alternative balloon catheter) is then advanced along the guidewire and at least partially through the distal open end 82 of the stent 80. The balloon member 8 is inflated as shown in FIG. 37 to expand at least that portion of the stent 80 that is located distal of the side opening 92 into engagement with the main vessel 72. In some arrangements, such as shown in FIG. 37, the balloon member 8 has sufficient length to span an entire length of the stent 80 from the proximal open end 84 to the distal open end 82. The balloon member 8 can provide a relatively consistent expansion of the stent 80 into engagement with the main vessel 72 both proximal and distal of the side opening 92.

The vessel bifurcation 70 can be additionally treated by, for example, deploying a branch stent that is positioned within the branch vessel 74 and at least partially overlapping the expandable structure 90 in the area of the ostium 76.

The various method systems and methods described above with reference to FIGS. 1-37 provide for treatment of a vessel bifurcation using a single guidewire. Using a single guidewire for treatment of a vessel bifurcation can reduce the complexity of the treatment process by avoiding problems that exist when using two or more guidewires (e.g., guidewire twist problems). Furthermore, the use of a balloon member having a bulge portion that extends around an entire circumference of the balloon member can have advantages as compared to the use of a balloon member having a bulge portion at a location along a length of the balloon member that must be aligned both radially and axially relative to the ostium of the branch vessel. Substantially eliminating the need for radial positioning of a bulge portion of a balloon for treatment of a vessel bifurcation can improve providing radially outward expansion of portions of the stent into the branch vessel.

### Materials and Other Considerations

A wide variety of stents, catheters, and guidewire configurations can be used with the catheter assembly embodiments of the present disclosure. The inventive principles disclosed herein should not be limited to any particular design or configuration. Some example stents that can be used with the catheter assemblies disclosed herein can be found in, for example, U.S. Pat. Nos. 6,210,429, 6,325,826, 6,706,062, and 7,220,275, the entire contents of which are incorporated herein by reference. In general, the aforementioned stents include a lateral branch opening located between distal and proximal open ends of the stent. The lateral branch opening defines a path between an inner lumen or inner volume of the stent and an area outside of the stent. The stent lateral branch opening is distinct from the cell openings defined between strut structures from which the stent sidewall is constructed. In some stents, the lateral branch opening can be surrounded by expandable structure. The expandable structure can be configured to extend radially into the branch lumen of the bifurcation upon expansion of, for example, an inflatable portion of the bifurcation treatment system. Typically, the stent is expanded after being positioned in the main lumen with the lateral branch opening aligned with an opening into the branch lumen. Alignment of the lateral branch opening with the opening into the branch lumen includes both radial and axial alignment. The stent, including the expandable structure surrounding the lateral branch opening, can be expanded with a single expansion or multiple expansions using one or more inflatable members.

The main and side balloons, and all other balloons disclosed herein, can be made of any suitable balloon material including compliant and non-compliant materials and combinations thereof. Some example materials for the balloons and catheters disclosed herein include thermoplastic polymers, polyethylene (high density, low density, intermediate density, linear low density), various copolymers and blends of polyethylene, ionomers, polyesters, polycarbonates, polyamides, poly-vinyl chloride, acrylonitrile-butadiene-styrene copolymers, polyether-polyester copolymers, and polyetherpolyamide copolymers. One suitable material is Surlyn®, a copolymer polyolefin material (DuPont de Nemours, Wilmington, Del.). Still further suitable materials include thermoplastic polymers and thermoset polymeric materials, poly(ethylene terephthalate) (commonly referred to as PET), thermoplastic polyamide, polyphenylene sulfides, polypropylene. Some other example materials include polyurethanes and block copolymers, such as polyamide-polyether block copolymers or amide-tetramethylene glycol copolymers. Additional examples include the PEBAX® (a polyamide/polyether/polyester block copolymer) family of polymers, e.g., PEBAX® 70D, 72D, 2533, 5533, 6333, 7033, or 7233 (available from Elf AtoChem, Philadelphia, Pa.). Other examples include nylons, such as aliphatic nylons, for example, Vestamid L21011F, Nylon 11 (Elf Atochem), Nylon 6 (Allied Signal), Nylon 6/10 (BASF), Nylon 6/12 (Ashley Polymers), or Nylon 12. Additional examples of nylons include aromatic nylons, such as Grivory (EMS) and Nylon MXD-6. Other nylons and/or combinations of nylons can also be used. Still further examples include polybutylene terephthalate (PBT), such as CELANEX® (available from Ticona, Summit, N.J.), polyester/ether block copolymers such as ARNITEL® (available from DSM, Erionspilla, Ind.), e.g., ARNITEL® EM740, aromatic amides such as Trogamid (PA6-3-T, Degussa), and thermoplastic elastomers such as HYTREL® (Dupont de Nemours, Wilmington, Del.). In some embodiments, the PEBAX®, HYTREL®, and ARNITEL® materials have a Shore D hardness of about 45D to about 82D. The balloon materials can be used pure or as blends. For example, a blend may include a PBT and one or more PBT thermoplastic elastomers, such as RITEFLEX® (available from Ticona), ARNITEL®, or HYTREL®, or polyethylene terephthalate (PET) and a thermoplastic elastomer, such as a PBT thermoplastic elastomer. Additional examples of balloon material can be found in U.S. Pat. No. 6,146,356, which is incorporated herein by reference.

The catheter assemblies described herein can include marker material that is visible under X-ray or in fluoroscopy procedures. For example, the marker material can be more easily identified and distinguished under X-ray or in fluoroscopy procedures. Some example marker materials include gold, platinum and tungsten. In one embodiment, the marker material can be included in a band structure that is secured to any portion of the catheter structure such as the balloon, catheter shaft, or guidewire housing. In other embodiments, the marker material is part of the material composition of portions of the catheter assembly. Viewability of features of the catheter assembly under X-ray or fluoroscopy can assist a physician operating the catheter assembly to more easily adjust a position of the assembly relative to the vessel bifurcation. Example markers and marker materials suitable for use with assembly 10 are described in, for example, U.S. Pat. No. 6,692,483 to Vardi, et al., and co-pending U.S. Published Patent Application No. 2007/0203562, filed on Feb. 22, 2007, and titled MARKER ARRANGEMENT FOR BIFURCATION CATHETER, which matters are incorporated herein by reference.

One aspect of the present disclosure relates to a catheter assembly that includes a balloon member and a balloon restricting arrangement. The balloon member includes a distal portion, a proximal portion, and a circumferential bulge portion. The circumferential bulge portion is positioned at a location between the proximal and distal portions of the balloon, and extends around a circumference of the balloon. The balloon restricting arrangement includes distal and proximal restricting members. The distal restricting member is configured to restrict a radially outward expanded dimension of the distal portion of the balloon to a restricted distal inflated dimension. The proximal restricting member is configured to restrict a radially outward expanded dimension of the proximal portion of the balloon to a restricted proximal inflated dimension. The circumferential bulge portion of the balloon has a maximum inflated dimension that is greater than the restricted proximal inflated dimension and the restricted distal inflated dimension.

Another aspect of the present disclosure relates to a balloon catheter that includes a catheter shaft, a balloon member, and a balloon restricting arrangement. The catheter shaft has a distal end portion and defines an inflation lumen. The balloon member is positioned at the distal end portion of the catheter shaft and in fluid communication with the inflation lumen. The balloon member includes a distal portion, a proximal portion, and a circumferential bulge portion. The balloon restricting arrangement includes distal and proximal restricting members. The distal restricting member is configured to restrict a radially outward expanded dimension of the distal portion of the balloon to a restricted distal inflated dimension. The proximal restricting member is configured to restrict a radially outward expanded dimension of the proximal portion of the balloon to a restricted proximal inflated dimension. The circumferential bulge portion of the balloon has a maximum inflated dimension that is greater than the restricted proximal inflated dimension and the restricted distal inflated dimension.

A still further aspect of the present disclosure relates to a method of expanding a stent with a post dilatation balloon catheter. The post dilatation balloon catheter includes a balloon member and a balloon restricting arrangement. The balloon member includes a proximal portion, a distal portion, and a circumferential bulge portion. The balloon restricting arrangement includes a distal restricting member and a proximal restricting member, wherein the distal restricting member is configured to restrict a radially outward expanded dimension of the distal portion of the balloon, and the proximal restricting member is configured to restrict a radially outward expanded dimension of the proximal portion. The circumferential bulge portion extends around a circumference of the balloon and has a maximum inflated dimension that is greater than the radially outward expanded dimension of the proximal and distal portions of the balloon. The method includes positioning the circumferential bulge portion in the stent, and inflating the balloon member such that the circumferential bulge portion expands a portion of the stent and the proximal and distal portions of the balloon do not expand the stent.

A further method aspect of the present disclosure relates to a method of treating a vessel bifurcation using a stent, a stent delivery catheter, and a post dilatation balloon catheter. The stent includes a first portion and a second portion, wherein the first portion of the stent has a strut density that is greater than a strut density of the second portion of the stent. The post dilatation balloon catheter includes a balloon member and a balloon restricting arrangement. The balloon member has a proximal portion, a distal portion, and a circumferential bulge portion. The balloon restricting arrangement includes a distal restricting member and a proximal restricting member, wherein the distal restricting member is configured to restrict a radially outward expanded dimension of the distal portion of the balloon to a restricted distal expanded dimension, and the proximal restricting member is configured to restrict a radially outward expanded dimension of the proximal portion to a restricted proximal expanded dimension. The circumferential bulge portion extends around a circumference of the balloon and has a maximum inflated dimension that is greater than the restricted distal expanded dimension and the restricted proximal expanded dimension. The method includes delivering the stent to the vessel bifurcation with the delivery catheter, wherein the stent is positioned with the second portion of the stent in axial alignment with an opening into a branch vessel of the vessel bifurcation. The method can also include expanding the stent into engagement with the main vessel using the delivery catheter, wherein the expanded stent has a maximum internal dimension, and positioning the balloon member of the post dilatation balloon catheter at least partially within the expanded stent with the circumferential bulge portion positioned in axial alignment with the second portion of the stent and the opening into the branch vessel. The method can further include inflating the balloon member of the post dilatation balloon catheter, wherein the maximum inflated dimension of the circumferential bulge portion is greater than the maximum internal dimension of the stent to expand at least a portion of the second portion of the stent into the branch vessel.

Another aspect of the present disclosure relates to a method of treating a vessel bifurcation with a catheter assembly. The catheter assembly includes a guidewire, a catheter branch, and a stent. The catheter branch including a balloon member having a main balloon portion and a bulge portion, wherein the bulge portion extends around a circumference of the main balloon portion and is positioned at a location between proximal and distal end portions of the main balloon portion. The bulge portion has a maximum diameter dimension that is greater than a maximum diameter dimension of the main balloon portion. The stent is positioned on the balloon member in alignment with the bulge portion. The method steps includes advancing the guidewire into a main vessel of the vessel bifurcation distally beyond an ostium of a branch vessel of the vessel bifurcation, advancing the catheter branch and stent over the guidewire to the vessel bifurcation with the bulge portion of the balloon member axially aligned with the ostium of the branch vessel, and inflating the balloon member to expand at least a portion of the stent through the ostium into the branch vessel. The method can also include retracting the catheter branch proximally, retracting the guidewire proximal of the ostium, advancing the guidewire through the portion of the expanded stent and into the branch vessel, advancing a post dilatation catheter over the guidewire and through the portion of the expanded stent, and further expanding the portion of the stent with the post dilatation catheter.

A still further aspect of the present disclosure relates to a method of treating a vessel bifurcation with a catheter assembly, wherein the catheter assembly includes a guidewire, a catheter branch, and a stent. The catheter branch includes a balloon member having a main balloon portion and a bulge portion, wherein the bulge portion extends around a circumference of the main balloon portion and is positioned at a location between proximal and distal end portions of the main balloon portion. The bulge portion has a maximum diameter dimension that is greater than a maximum diameter dimension of the main balloon portion. The method can include advancing the guidewire into a main vessel of the vessel bifurcation distally beyond an ostium of a branch vessel of the vessel bifurcation, positioning the stent in the main vessel spanning the ostium, expanding the stent into engagement with the main vessel, and advancing the catheter branch over the guidewire to the vessel bifurcation with the bulge portion of the balloon member axially aligned with the ostium of the branch vessel. The method can further include inflating the balloon member to expand at least a portion of the stent through the ostium into the branch vessel, retracting the catheter branch proximally, retracting the guidewire proximal of the ostium, advancing the guidewire through the portion of the expanded stent and into the branch vessel, advancing a post dilatation catheter over the guidewire and through the portion of the expanded stent, and further expanding the portion of the stent with the post dilatation catheter.

It is noted that not all of the features characterized herein need to be incorporated within a given arrangement, for the arrangement to include improvements according to the present disclosure.

1. A catheter assembly, comprising:
(a) a balloon member having a distal portion, a proximal portion, and a circumferential bulge portion, the circumferential bulge portion being positioned at a location between the proximal and distal portions of the balloon, the circumferential bulge portion extending around a circumference of the balloon; and
(b) a balloon restricting arrangement, the balloon restricting arrangement including:
i. a distal restricting member configured to restrict a radially outward expanded dimension of the distal portion of the balloon to a restricted distal inflated dimension; and
ii. a proximal restricting member configured to restrict a radially outward expanded dimension of the proximal portion of the balloon to a restricted proximal inflated dimension, the circumferential bulge portion of the balloon having a maximum inflated dimension that is greater than the restricted proximal inflated dimension and the restricted distal inflated dimension.

2. The assembly of claim 1, further comprising:
(a) a catheter shaft having a distal end portion; and
(b) a guidewire housing extending within at least a portion of the catheter shaft, a portion of the guidewire housing extending distally beyond the distal end portion of the catheter shaft;
(c) wherein the proximal portion of the balloon member is mounted to the distal end portion of the catheter shaft, and the distal portion of the balloon member is mounted to the portion of the guidewire housing that extends distally beyond the distal end portion of the catheter shaft.

3. The assembly of claim 1, wherein at least one of the distal restricting member and the proximal restricting member includes a sleeve portion, the sleeve portion comprising material that is different from a material of the balloon member.

4. The assembly of claim 1, wherein at least one of the distal restricting member and the proximal restricting member includes a sleeve portion, the sleeve portion comprising material that is the same as a material of the balloon member.

5. The assembly of claim 1, wherein the distal and proximal restricting members are secured to the balloon member.

6. The assembly of claim 1, wherein at least one of the distal restricting member and the proximal restricting member includes a plurality of fiber members that extend around at least a portion of the circumference of the balloon member.

7. The assembly of claim 1, wherein at least one of the distal restricting member and the proximal restricting member includes a plurality of first fiber members and the circumferential bulge portion includes a plurality of second fiber members, wherein the first fiber members have a length that is less than a length of the second fiber members.

8. The assembly of claim 1, wherein the circumferential bulge portion includes a plurality of fiber members, wherein the plurality of fiber members restrict a radially outward expanded dimension of the circumferential bulge portion to the maximum inflated dimension.

9. A balloon catheter, comprising:
(a) a catheter shaft having a distal end portion and defining an inflation lumen;
(b) a balloon member positioned at the distal end portion of the catheter shaft and in fluid communication with the inflation lumen, the balloon member including a distal portion, a proximal portion, and a circumferential bulge portion; and
(c) a balloon restricting arrangement, the balloon restricting arrangement including:
i. a distal restricting member configured to restrict a radially outward expanded dimension of the distal portion of the balloon to a restricted distal inflated dimension; and
ii. a proximal restricting member configured to restrict a radially outward expanded dimension of the proximal portion of the balloon to a restricted proximal inflated dimension, the circumferential bulge portion of the balloon having a maximum inflated dimension that is greater than the restricted proximal inflated dimension and the restricted distal inflated dimension.

10. The balloon catheter of claim 9, wherein at least one of the distal restricting member and the proximal restricting member comprises a material that is different from a material of the balloon member.

11. The balloon catheter of claim 9, wherein at least one of the distal restricting member and the proximal restricting member comprises a material that is the same as a material of the balloon member.

12. The balloon catheter of claim 9, wherein the distal and proximal restricting members are secured to the balloon member.

13. The balloon catheter of claim 9, wherein at least one of the distal restricting member and the proximal restricting member includes a plurality of first fiber members, wherein each of the plurality of first fiber members extends around the circumference of the balloon member.

14. The balloon catheter of claim 13, wherein the circumferential bulge portion includes a plurality of second fiber members, wherein the plurality of second fiber members extend around the circumference of the balloon member, wherein the first fiber members have a length that is less than a length of the second fiber members.

15. The balloon catheter of claim 9, wherein the distal and proximal restricting members are connected together with at plurality of connecting strips that extend from a distal end of the proximal restricting member to a proximal end of the distal restricting member.

16. A method of expanding a stent with a post dilatation balloon catheter, the post dilatation balloon catheter including a balloon member and a balloon restricting arrangement, the balloon member having a proximal portion, a distal portion, and a circumferential bulge portion, the balloon restricting arrangement including a distal restricting member and a proximal restricting member, the distal restricting member being configured to restrict a radially outward expanded dimension of the distal portion of the balloon, and the proximal restricting member is configured to restrict a radially outward expanded dimension of the proximal portion, the circumferential bulge portion extending around a circumference of the balloon and having a maximum inflated dimension that is greater than the radially outward expanded dimension of the proximal and distal portions of the balloon, the method comprising:
(a) positioning the circumferential bulge portion in the stent; and
(b) inflating the balloon member, wherein the circumferential bulge portion expands a portion of the stent, and the proximal and distal portions of the balloon do not expand the stent.

17. The method of claim 16, further comprising at least partially expanding the stent with a delivery balloon catheter prior to the step of positioning the circumferential bulge portion in the stent.

18. A method of treating a vessel bifurcation using a stent, a stent delivery catheter, and a post dilatation balloon catheter, the stent including a first portion and a second portion, the first portion of the stent having a strut density that is greater than a strut density of the second portion of the stent, the post dilatation balloon catheter including a balloon member and a balloon restricting arrangement, the balloon member having a proximal portion, a distal portion, and a circumferential bulge portion, the balloon restricting arrangement including a distal restricting member and a proximal restricting member, the distal restricting member is configured to restrict a radially outward expanded dimension of the distal portion of the balloon to a restricted distal expanded dimension, and the proximal restricting member is configured to restrict a radially outward expanded dimension of the proximal portion to a restricted proximal expanded dimension, the circumferential bulge portion extending around a circumference of the balloon and having a maximum inflated dimension that is greater than the restricted distal expanded dimension and the restricted proximal expanded dimension, the method comprising:
(a) delivering the stent to the vessel bifurcation with the delivery catheter, the stent being positioned with the second portion of the stent in axial alignment with an opening into a branch vessel of the vessel bifurcation;
(b) expanding the stent into engagement with the main vessel using the delivery catheter, the expanded stent having a maximum internal dimension;
(c) positioning the balloon member of the post dilatation balloon catheter at least partially within the expanded stent with the circumferential bulge portion positioned in axial alignment with the second portion of the stent and the opening into the branch vessel; and
(d) inflating the balloon member of the post dilatation balloon catheter, wherein the maximum inflated dimension of the circumferential bulge portion is greater than the maximum internal dimension of the stent to expand at least a portion of the second portion of the stent into the branch vessel.

19. The method of claim 18, wherein the expanded stent has a minimum internal dimension, and the restricted distal expanded dimension and the restricted proximal expanded dimension of the balloon member are less than the minimum internal dimension of the stent.

20. The method of claim 18, wherein inflating the balloon member of the post dilatation balloon catheter includes first inflating the proximal and distal portions of the balloon member into engagement with the proximal and distal restricting members, respectively, followed by inflating the circumferential bulge portion to the maximum inflated dimension.

21. The method of claim 18, further comprising retracting the post dilatation balloon catheter from the stent and advancing a post dilatation balloon through that portion of the stent that is expanded into the branch vessel, and inflating the post dilatation balloon to expand a portion of the stent into engagement with the branch vessel.

22. A method of treating a vessel bifurcation with a catheter assembly, the catheter assembly including a guidewire, a catheter branch, and a stent, the catheter branch including a balloon member having a main balloon portion and a bulge portion, the bulge portion extending around a circumference of the main balloon portion and positioned at a location between proximal and distal end portions of the main balloon portion, the bulge portion having a maximum diameter dimension that is greater than a maximum diameter dimension of the main balloon portion, the stent positioned on the balloon member in alignment with the bulge portion, the method comprising:
(a) advancing the guidewire into a main vessel of the vessel bifurcation distally beyond an ostium of a branch vessel of the vessel bifurcation;
(b) advancing the catheter branch and stent over the guidewire to the vessel bifurcation with the bulge portion of the balloon member axially aligned with the ostium of the branch vessel;
(c) inflating the balloon member to expand at least a portion of the stent through the ostium into the branch vessel;
(d) retracting the catheter branch proximally;
(e) retracting the guidewire proximal of the ostium;
(f) advancing the guidewire through the portion of the expanded stent and into the branch vessel;
(g) advancing a post dilatation catheter over the guidewire and through the portion of the expanded stent; and
(h) further expanding the portion of the stent with the post dilatation catheter.

23. A method of treating a vessel bifurcation with a catheter assembly, the catheter assembly including a guidewire, a catheter branch, and a stent, the catheter branch including a balloon member having a main balloon portion and a bulge portion, the bulge portion extending around a circumference of the main balloon portion and positioned at a location between proximal and distal end portions of the main balloon portion, the bulge portion having a maximum diameter dimension that is greater than a maximum diameter dimension of the main balloon portion, the method comprising:
(a) advancing the guidewire into a main vessel of the vessel bifurcation distally beyond an ostium of a branch vessel of the vessel bifurcation;
(b) positioning the stent in the main vessel spanning the ostium;
(c) expanding the stent into engagement with the main vessel;
(d) advancing the catheter branch over the guidewire to the vessel bifurcation with the bulge portion of the balloon member axially aligned with the ostium of the branch vessel;
(e) inflating the balloon member to expand at least a portion of the stent through the ostium into the branch vessel;
(f) retracting the catheter branch proximally;
(g) retracting the guidewire proximal of the ostium;
(h) advancing the guidewire through the portion of the expanded stent and into the branch vessel;
(i) advancing a post dilatation catheter over the guidewire and through the portion of the expanded stent; and
(j) further expanding the portion of the stent with the post dilatation catheter.
